(19) United States
(12) Patent Application Publication
(10) Pub. No.: US 2025/0305194 A1
(43) Pub. Date: Oct. 2, 2025

(54) NON-WOVEN FABRIC WIPE AND MANUFACTURING METHOD THEREFOR

(71) Applicant: XIAMEN YANJAN NEW MATERIAL CO., LTD, Fujian (CN)

(72) Inventors: Shihuang LI, Fujian (CN); Yonggong CHEN, Fujian (CN); Jinyi JI, Fujian (CN)

(21) Appl. No.: 18/867,020

(22) PCT Filed: Aug. 17, 2022
(86) PCT No.: PCT/CN2022/113033

§ 371 Date: Nov. 19, 2024

(30) Foreign Application Priority Data
Jun. 24, 2022 (CN) 202210730207.9

(51) Int. Cl. D04H 1/56; A61K 8/02; D04H 1/26

(52) U.S. Cl. CPC D04H 1/565; A61K 8/0208; D04H 1/26; A61K 2800/622

(57) ABSTRACT

A non-woven fabric wipe having a layered fiber web structure. A first layer, a second layer, a fourth layer, and a fifth layer of the non-woven fabric wipe are all melt-blown fiber webs mainly composed of melt-blown fibers; a third layer of the non-woven fabric wipe is a wood pulp fiber web mainly composed of wood pulp fibers; the melt-blown fibers in the melt-blown fiber webs of the second layer and the fourth layer are partially interwoven in the adjacent wood pulp fiber web of the third layer, Because the non-woven fabric wipe of the present invention is composed of five layers of fiber webs, when the melt-blown fiber webs of the second layer and the fourth layer are consolidated, the portions interwoven with the wood pulp fibers are consolidated together with the interwoven wood pulp fibers, thereby forming a protective web that prevents the wood pulp fibers from moving.

### TECHNICAL FIELD

The present invention relates to the technical field of non-woven fabric wipes, and in particular to an abrasion-resistant and lint-free non-woven fabric wipe applied in personal care and infant care, and a manufacturing method therefor.

### BACKGROUND OF THE INVENTION

At present, wiping cotton soft towels have gained popularity among consumers due to their convenience in carrying and storage, and ease of use. The wiping cotton soft towels can be spunlace non-woven fabric products or melt-blown composite non-woven fabric products. Compared to traditional cloth or paper soft towels, the production of wiping cotton soft towels is convenient and cost-effective, and these towels can be used in both dry and wet conditions.

However, the existing melt-blown composite non-woven fabric is formed by compounding melt-blown fiber layers serving as surface layers and a wood pulp fiber layer serving as a middle layer, and the wood pulp fibers are short in length and easily drop from the melt-blown fiber layers, that is, the so-called phenomenon of “linting”. The wood pulp fibers dropped in a wiping process will remain on the surface of the object concerned, resulting in poor cleaning.

### BRIEF SUMMARY OF THE INVENTION

### Technical Problems to be Solved

It is an object of the present invention to provide an abrasion-resistant and lint-free non-woven fabric wipe and a manufacturing method therefor, thereby overcoming the defects of existing products and production methods.

### Technical Solutions to the Technical Problems

### Technical Solutions

To achieve the aforementioned object, the present invention provides the following technical solutions: A non-woven fabric wipe having a layered fiber web structure, comprising a first layer, a second layer, a third layer, a fourth layer and a fifth layer which are sequentially arranged; the first layer, the second layer, the fourth layer and the fifth layer of the non-woven fabric wipe are all melt-blown fiber webs mainly composed of melt-blown fibers, and the third layer of the non-woven fabric wipe is a wood pulp fiber web mainly composed of wood pulp fibers, wherein the first layer and the fifth layer each has a melt-blown fiber denier of 0.2-0.4 denier, the second layer and the fourth layer each has a melt-blown fiber denier of 0.5-2.0 denier, and a weight percentage of the third layer to the non-woven fabric wipe is ≥65%; the melt-blown fibers in the melt-blown fiber webs of the second layer and the fourth layer are partially interwoven into the wood pulp fiber web of the third layer adjacent thereto.

The melt-blown fibers are polyolefin fibers, polyester fibers, polyamide fibers, polyurethane fibers, polylactic acid fibers, or a mixture thereof.

The melt-blown fibers are single-component melt-blown fibers, bi-component melt-blown fibers, or melt-blown fibers with a blend of the single-component melt-blown fibers and the bi-component melt-blown fibers.

The bi-component melt-blown fibers are composed of fibers having a melting point difference of 20° C. or more, and each of the fibers having low-melting point resin at its surface; the bi-component melt-blown fibers are sheath-core type, pie-segmented type or side-by-side type melt-blown fibers.

The wood pulp fiber web of the third layer is composed of a blend of wood pulp fibers and other fibers or substances.

The other fibers are viscose fibers, single-component or bi-component chemical fibers, or other natural fibers or mixed fibers thereof.

The other substances are hot-melt adhesive substances or super absorbent resins.

An embossed area is formed on a surface of the non-woven fabric wipe.

A hydrophilic coating or a functional coating is provided on an upper surface of the melt-blown fiber web of the first layer.

The present invention further provides a manufacturing method for manufacturing the aforementioned non-woven fabric wipe, comprising steps of:
(1) pulverizing a pulp sheet with a pulverizer to form a wood pulp fiber web which enters a first melt-blown compounding procedure through a nozzle under action of an auxiliary airflow;
(2) performing the first melt-blown compounding procedure, where a first and a second melt-blown spinning devices are positioned at two sides of the wood pulp fiber web respectively; by using a melt-blowing process, a thermoplastic resin is heated, and enters the first and the second melt-blown spinning devices after melted; molten fine flows of the thermoplastic resin in the first and the second melt-blown spinning devices are ejected from melt-blown spinneret orifices of melt-blown spinneret plates and blown into fiber bundles each with a fiber diameter of ≤10 μm by using hot airflows, and thus melt-blown fiber webs are formed with the hot airflows; each of the melt-blown fiber webs formed is intersected with a side surface of the wood pulp fiber web that is adjacent thereto with an intersection included angle therebetween being 15°-60°, thereby forming a multilayer structured fiber web with melt-blown fiber webs on two sides and a wood pulp fiber web therebetween, with the multilayer structured fiber web corresponding to the second layer, the third layer and the fourth layer of the non-woven fabric wipe;
(3) conveying the multilayer structured fiber web to undergo a second melt-blown compounding procedure, where a first layer of melt-blown fiber web is attached to an upper surface of the multilayer structured fiber web via a third melt-blown spinning device by using a melt-blowing process, thereby forming the first layer of the non-woven fabric wipe, and a compounding included angle between the first layer of the non-woven fabric wipe and the multilayer structured fiber web is 90°;
(4) conveying the multilayer structured fiber web attached with the first layer of melt-blown fiber web on the upper surface thereof to undergo a third melt-blown compounding procedure, where a second layer of melt-blown fiber web is attached to a lower surface of the multilayer structured fiber web via a fourth melt-blown spinning device by using a melt-blowing process, thereby forming the fifth layer of the non-woven fabric wipe, and a compounding included angle between the fifth layer of the non-woven fabric wipe and the multilayer structured fiber web is 90°;
(5) performing a consolidation procedure, where the first layer, the second layer, the third layer, the fourth layer and the fifth layer of the non-woven fabric wipe are consolidated together via a heating apparatus to form the non-woven fabric wipe.

The melt-blown spinneret orifices are single-component spinneret orifices, bi-component spinneret orifices, or a mixed arrangement thereof.

The bi-component spinneret orifices are of sheath-core type, pie-segmented type, or side-by-side type.

The hearing apparatus is a hot air oven, hot rollers, or a combination thereof.

A post-treatment procedure is performed after the consolidation procedure, where the first layer of the non-woven fabric wipe is coated with a hydrophilic coating or a function coating by means of spray coating or roll coating.

### Beneficial Effects of the Present Invention

### Beneficial Effects

If the aforementioned technical solutions are adopted, since the non-woven fabric wipe of the present invention is composed of five layers of fiber webs, where the third layer is wood pulp fibers, the first layer, the second layer, the fourth layer and the fifth layer are all composed of melt-blown fibers, and the melt-blown fibers of the second layer, the third layer and the fourth layer positioned at the middle position are partially interwoven with the wood pulp fibers; when the melt-blown fiber webs of the second layer and the fourth layer are consolidated, the portions interwoven with the wood pulp fibers will be consolidated together with the interwoven wood pulp fibers. The melt-blown fiber deniers of the second layer and the fourth layer are 0.5-2.0 denier, and the fibers are thicker and are easier to interweave with the wood pulp. The consolidated wood pulp fibers are positioned at the upper and lower surfaces of the third layer, such that a protective web that prevents the wood pulp fibers from moving is formed, and the melt-blown fiber webs of the first layer, the second layer, the fourth layer and the fifth layer positioned outside the third layer protect the wood pulp fibers layer by layer, thereby preventing the phenomenon of “linting”. The melt-blown fiber webs of the first layer and the fifth layer are compounded with the multilayer structured fiber web with compounding included angles all being 90°, such that the melt-blown fiber webs of the first layer and the fifth layer are not in contact with the non-melt-blown fibers, and therefore the formed melt-blown fiber webs only contain melt-blown fibers. The fiber deniers of the first layer and the fifth layer are 0.2-0.4 denier, and the fibers are finer. A more compact and uniform fiber structure can be formed to prevent flake dropping and linting, and is more favorable for the protection of the wood pulp short fibers in the third layer, and the overall soft touch of the non-woven fabric wipe is also improved. Furthermore, the melt-blown fiber surface of the first layer positioned at the surface layer of the non-woven fabric wipe may be further subjected to a hydrophilic post-treatment. Meanwhile, the fiber thicknesses and densities of the first layer and the fifth layer can be adjusted to meet different requirements of clients and to be applied in different fields.

### BRIEF DESCRIPTION OF THE DRAWINGS

### Description of the Drawings

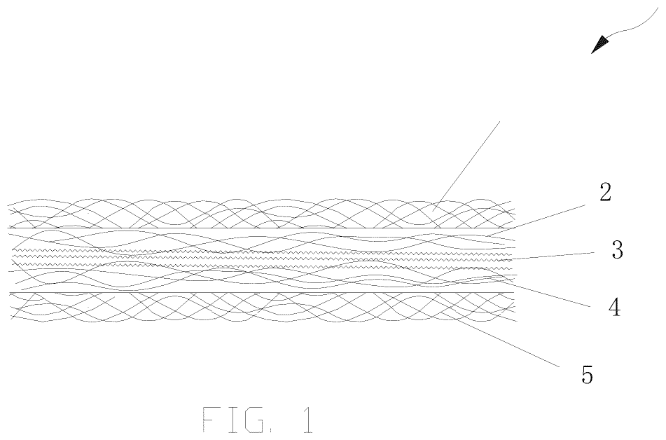

FIG. 1 is a cross-sectional view of the non-woven fabric wipe according to Embodiment 1 of the present invention;

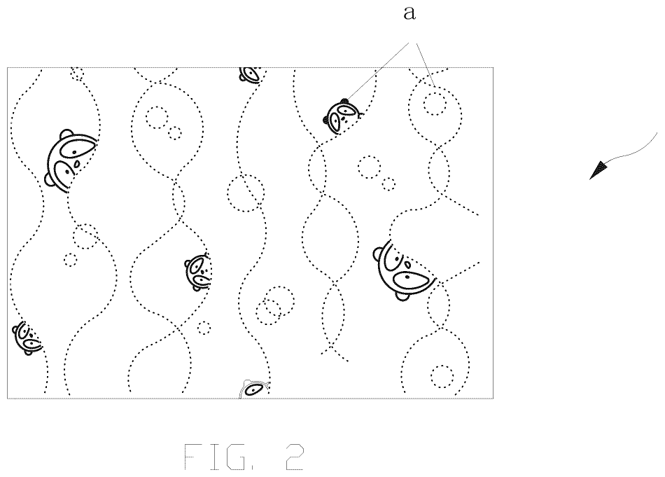

FIG. 2 is a top view of the non-woven fabric wipe according to Embodiment 1 of the present invention;

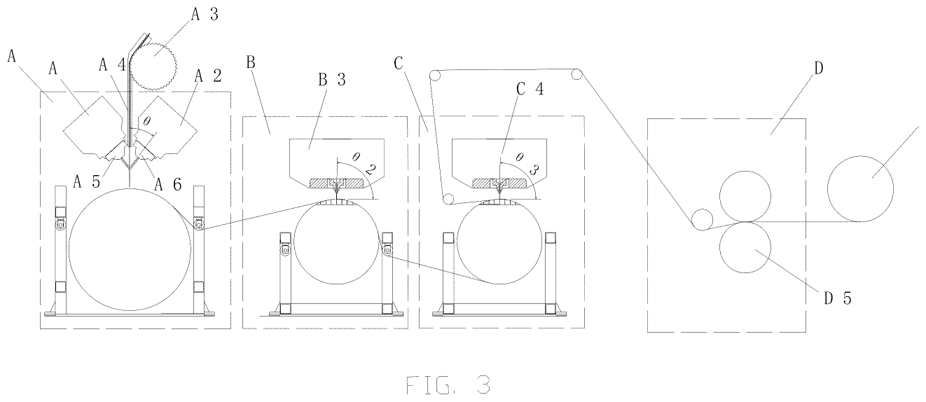

FIG. 3 is a schematic diagram of the manufacturing of the non-woven fabric wipe according to Embodiment 1 of the present invention;

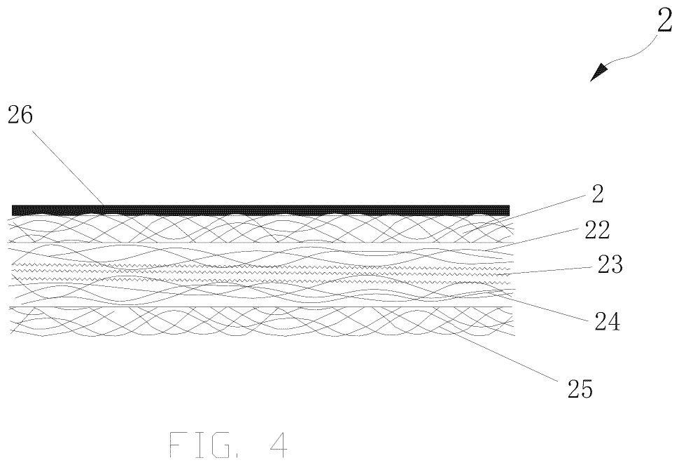

FIG. 4 is a cross-sectional view of the non-woven fabric wipe according to Embodiment 2 of the present invention;

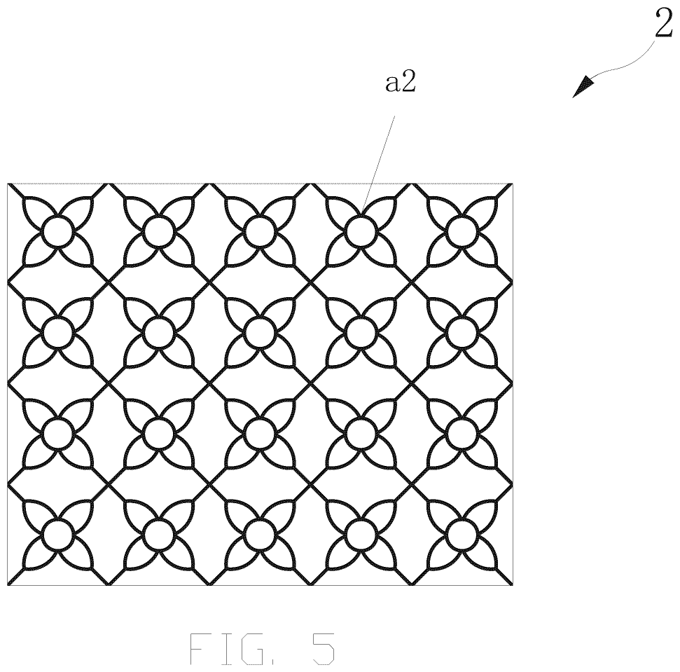

FIG. 5 is a top view of the non-woven fabric wipe according to Embodiment 2 of the present invention; and

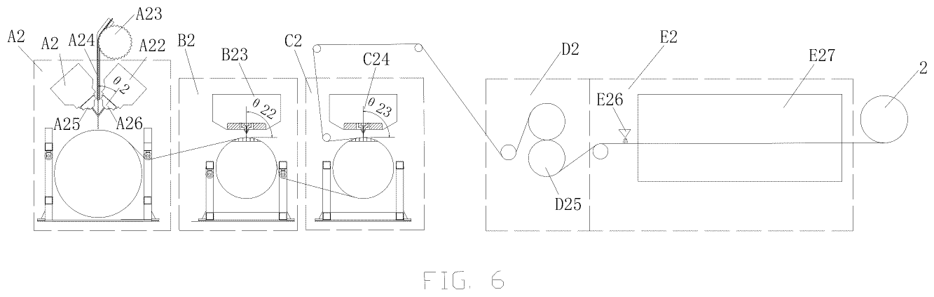

FIG. 6 is a schematic diagram of the manufacturing of the non-woven fabric wipe according to Embodiment 2 of the present invention.

### DESCRIPTION OF REFERENCE NUMERALS

### Embodiment 1

A non-woven fabric wipe 1
A first layer 11; a second layer 12; a third layer 13; fourth layer 14; a fifth layer 15
An embossed area a1
A first melt-blown compounding procedure A1
A first melt-blown spinning device A11; a second melt-blown spinning device A12
A pulverizer A13; a nozzle A14; melt-blown spinneret plates A15, A16
A second melt-blown compounding procedure B1; a third melt-blown spinning device B13
A third melt-blown compounding procedure C1; a fourth melt-blown spinning device C14
A consolidation procedure D1; a hot roller D15

### Embodiment 2

A non-woven fabric wipe 2
A first layer 21; a second layer 22; a third layer 23; a fourth layer 24; a fifth layer 25
An embossed area a2; a hydrophilic coating 26
A first melt-blown compounding procedure A2
A first melt-blown spinning device A21; a second melt-blown spinning device A22
A pulverizer A23; a nozzle A24; melt-blown spinneret plates A25, A26
A second melt-blown compounding procedure B2; a third melt-blown spinning device B23
A third melt-blown compounding procedure C2; a fourth melt-blown spinning device C24
A consolidation procedure D2; a hot roller D25
A post-treatment procedure E2; a spray coating apparatus E26; an oven E27

### EMBODIMENTS OF THE INVENTION

### Embodiments of the Present Invention

To further explain the technical solutions of the present invention, a detailed description of the present invention is provided below through specific embodiments.

### Embodiment 1

As shown in FIGS. 1 and 2, the present invention discloses a non-woven fabric wipe 1 having a layered fiber web structure. A first layer 11, a second layer 12, a fourth layer 14, and a fifth layer 15 of the non-woven fabric wipe 1 are all melt-blown fiber webs composed of polypropylene melt-blown fibers, and a third layer 13 of the non-woven fabric wipe 1 is a wood pulp fiber web composed of wood pulp fibers, wherein the first layer and the fifth layer each has a melt-blown fiber denier of 0.2-0.4 denier, the second layer and the fourth layer each has a melt-blown fiber denier of 0.5-2.0 denier, and a weight percentage of the third layer 13 to the non-woven fabric wipe 1 is 65%; the melt-blown fibers in the melt-blown fiber webs of the second layer 12 and the fourth layer 14 are partially interwoven into the wood pulp fiber web of the adjacent third layer 13, and an embossed area a1 is formed on the surface of the non-woven fabric wipe 1.

Melt-blown fibers of the non-woven fabric wipe 1 may be polyolefin fibers, polyester fibers, polyamide fibers, polyurethane fibers, polylactic acid fibers, or a mixture thereof, and the wood pulp fiber web of the third layer 13 is composed of a blend of wood pulp fibers and other fibers or substances. Other fibers are viscose fibers, single-component or bi-component chemical fibers, or other natural fibers or mixed fibers thereof; other substances are hot-melt adhesive substances or super absorbent resins.

As shown in FIG. 3, the present invention discloses a manufacturing method for the non-woven fabric wipe 1, which comprises the following steps:
(1) Pulverizing a pulp sheet with a pulverizer A13 to form a wood pulp fiber web which enters a first melt-blown compounding procedure A1 through a nozzle A14 under action of an auxiliary airflow.
(2) Performing the first melt-blown compounding procedure A1, where a first and a second melt-blown spinning devices A11, A12 are positioned at two sides of the wood pulp fiber web; by using a melt-blowing process, a thermoplastic resin polypropylene is heated, and enters the first and the second melt-blown spinning devices A11, A12 after melted. Molten fine flows of the thermoplastic resin in the first and the second melt-blown spinning devices A11, A12 are ejected from melt-blown spinneret orifices of melt-blown spinneret plates A15, A16 and blown into fiber bundles each with a fiber diameter of ≤10 μm by using hot airflows, and thus melt-blown fiber webs are formed with the hot airflows. Each of the melt-blown fiber webs formed is intersected with a side surface of the wood pulp fiber web that is adjacent thereto with an intersection included angle θ11 between the melt-blown fiber web and the wood pulp fiber web being 15°-60°, thereby forming a multilayer structured fiber web with melt-blown fiber webs on two sides and a wood pulp fiber web therebetween, corresponding to the second layer 12, the third layer 13 and the fourth layer 14 of the non-woven fabric wipe 1.
(3) Conveying the multilayer structured fiber web to undergo a second melt-blown compounding procedure B1, where a first layer of melt-blown fiber web is attached to an upper surface of the multilayer structured fiber web via a third melt-blown spinning device B13 by using a melt-blowing process, thereby forming the first layer 11 of the non-woven fabric wipe, and a compounding included angle θ12 between the first layer 11 of the non-woven fabric wipe 1 and the multilayer structured fiber web is 90°.
(4) Conveying the multilayer structured fiber web attached with the first layer of melt-blown fiber web on the upper surface thereof to undergo a third melt-blown compounding procedure C1, where a second layer of melt-blown fiber web is attached to a lower surface of the multilayer structured fiber web via a fourth melt-blown spinning device C14 by using a melt-blowing process, thereby forming the fifth layer 15 of the non-woven fabric wipe 1, and a compounding included angle θ13 between the fifth layer 15 of the non-woven fabric wipe 1 and the multilayer structured fiber web is 90°.
(5) Performing a consolidation procedure D1, where the first layer 11, the second layer 12, the third layer 13, the fourth layer 14 and the fifth layer 15 of the non-woven fabric wipe 1 are consolidated together via a heating apparatus, i.e., a pair of hot rollers D15, to form the non-woven fabric wipe 1.

The structure type of the melt-blown spinneret orifices can be single-component spinneret orifices, bi-component spinneret orifices, or a mixed arrangement thereof. The bi-component spinneret orifices can be of sheath-core type, pie-segmented type, or side-by-side type. The bi-component melt-blown fibers can be composed of fibers having a melting point difference of 20° C. or more, and each of the fibers having low-melting point resins at its surface. The melt-blown fibers formed in this way are single-component melt-blown fibers, bi-component melt-blown fibers, or melt-blown fibers with a blend of the single-component melt-blown fibers and the bi-component melt-blown fibers.

### Embodiment 2

As shown in FIGS. 4 and 5, the present invention discloses a non-woven fabric wipe 2 having a layered fiber web structure. A first layer 21, a second layer 22, a fourth layer 24, and a fifth layer 25 of the non-woven fabric wipe 2 are all melt-blown fiber webs composed of polypropylene melt-blown fibers, and a third layer 23 of the non-woven fabric wipe 2 is a wood pulp fiber web composed of wood pulp fibers; a weight percentage of the third layer 23 to the non-woven fabric wipe 1 is 80%; the melt-blown fibers in the melt-blown fiber webs of the second layer 22 and the fourth layer 24 are partially interwoven into the wood pulp fiber web of the third layer 23 adjacent thereto, an embossed area a2 is formed on the surface of the non-woven fabric wipe 1, and a hydrophilic coating 26 is further provided on the upper surface of the melt-blown fiber web of the first layer.

Melt-blown fibers of the non-woven fabric wipe 2 may be polyolefin fibers, polyester fibers, polyamide fibers, polyurethane fibers, polylactic acid fibers, or a mixture thereof, and the wood pulp fiber web of the third layer 23 is composed of a blend of wood pulp fibers and other fibers or substances. Other fibers are viscose fibers, single-component or bi-component chemical fibers, or other natural fibers or mixed fibers thereof; other substances are hot-melt adhesive substances or super absorbent resins.

As shown in FIG. 6, the present invention discloses a manufacturing method for the non-woven fabric wipe 2, which comprises the following steps:
(1) Pulverizing a pulp sheet with a pulverizer A23 to form a wood pulp fiber web which enters a first melt-blown compounding procedure A2 through a nozzle A24 under action of an auxiliary airflow.
(2) Performing the first melt-blown compounding procedure A2, where a first and a second melt-blown spinning devices A21, A22 are positioned at two sides of the wood pulp fiber web; by using a melt-blowing process, a thermoplastic resin polypropylene is heated, and enters the first and the second melt-blown spinning devices A21, A22 after melted. Molten fine flows of the thermoplastic resin in the first and the second melt-blown spinning devices A21, A22 are ejected from melt-blown spinneret orifices of melt-blown spinneret plates A25, A26 and blown into fiber bundles each with a fiber diameter of ≤10 μm by using hot airflows, and thus melt-blown fiber webs are formed with the hot airflows. Each of the melt-blown fiber webs formed is intersected with a side surface of the wood pulp fiber web that is adjacent thereto with an intersection included angle θ21 between the melt-blown fiber web and the wood pulp fiber web being 15°-60°, thereby forming a multilayer structured fiber web with melt-blown fiber webs on two sides and a wood pulp fiber web therebetween, corresponding to the second layer 22, the third layer 23 and the fourth layer 24 of the non-woven fabric wipe 2.
(3) Conveying the multilayer structured fiber web to undergo a second melt-blown compounding procedure B2, where a first layer of melt-blown fiber web is attached to an upper surface of the multilayer structured fiber web via a third melt-blown spinning device B23 by using a melt-blowing process, thereby forming the first layer 21 of the non-woven fabric wipe, and a compounding included angle θ22 between the first layer 21 of the non-woven fabric wipe 2 and the multilayer structured fiber web is 90°.
(4) Conveying the multilayer structured fiber web attached with the first layer of melt-blown fiber web on the upper surface thereof to undergo a third melt-blown compounding procedure C2, where a second layer of melt-blown fiber web is attached to a lower surface of the multilayer structured fiber web via a fourth melt-blown spinning device C24 by using a melt-blowing process, thereby forming the fifth layer 25 of the non-woven fabric wipe 2, and a compounding included angle θ23 between the fifth layer 25 of the non-woven fabric wipe 2 and the multilayer structured fiber web is 90°.
(5) Performing a consolidation procedure D2, where the first layer 21, the second layer 22, the third layer 23, the fourth layer 24 and the fifth layer 25 of the non-woven fabric wipe 2 are consolidated together via a heating apparatus, i.e., a pair of hot rollers D25.
(6) Performing a post-treatment procedure E2, where the first layer 21 of the non-woven fabric wipe 2 is coated with a hydrophilic coating 26 via a spray coating apparatus E26, and then heated and dried via an oven E27, to obtain the non-woven fabric wipe 2 of the present embodiment.

The structure type of the melt-blown spinneret orifices can be single-component spinneret orifices, bi-component spinneret orifices, or a mixed arrangement thereof. The bi-component spinneret orifices can be of sheath-core type, pie-segmented type, or side-by-side type. The melt-blown fibers formed in this way are single-component melt-blown fibers, bi-component melt-blown fibers, or melt-blown fibers with a blend of the single-component melt-blown fibers and the bi-component melt-blown fibers.

### Powder Drop Rate Testing

Instruments: powder drop rate tester, balance
Reference testing standard: Appendix B, Determination of Powder Drop Rate in GB/T 20810-2018 “Tissue Paper”

### Testing Steps:

1. Take approximately 150 g of the sample, weigh it with the balance, and denote its weight as m1; fold the sample into a specimen of 200 mm in length, with the long edge remaining level during folding.
2. Fix one end of the long edge of the specimen onto the specimen clamp, with the specimen surface perpendicular to the swinging direction during fixing, and ensure that the specimen does not come into contact with the inner wall of the chamber during the test.
3. Start the instrument and let the specimen swing inside the chamber for 2 min, with a reciprocating frequency of 180±10 times/min and a swing distance of 100±5 mm.
4. After the test is completed, turn off the instrument, remove the specimen, weigh the specimen mass and denote it as m2.
5. Calculate the powder drop rate of the specimen according to the following formula:

$$X = [ ( m 1 - m 2 ) \div m 1 ] \times 100$$

In the formula:
X denotes the powder drop rate of the specimen (%);
m1 denotes the mass of the specimen before treatment in grams (g);
m2 denotes the mass of the specimen after treatment in grams (g).

### Abrasion Resistance Testing

Reference standard GB/T13775-92 “Test Method for Abrasion Resistance of Cotton, Linen, and Spun Silk Woven Fabrics”
Instrument: YG(B)401E Martindale abrasion tester

### Materials Used in the Testing:

Standard padding: standard felt with a square meter weight of 750±50 g/m², thickness of 3±0.5 mm, and diameter of 140 mm.

Specimen backing material: polyurethane foam plastic with a thickness of 3±0.5 mm, density of 0.04 g/cm³, and diameter of 38±2 mm.

Sampler 1: disk sampler with a diameter of 140 mm, used for sampling the lower-layer abrasive material with a size of @140 mm.

Sampler 2: disk sampler with a diameter of 38 mm, used for sampling the upper-layer abrasive material with a size of φ38 mm.

Sample pretreatment: samples are kept at room temperature for 24 H.

### Testing Procedure:

1) Use sampler 1 to collect a lower-layer abrasive material with a diameter of 140 mm and place it over the standard padding; then place the sample loading hammer on the lower-layer abrasive material and tighten the circular clamp to fix the abrasive material on the specimen table.
2) Use sampler 2 to collect a specimen with a diameter of 38 mm and load the specimen with a sample holder into a 200 g A-type friction head metal clamp head, with a polyurethane foam plastic with a diameter of 38 mm inserted between the metal clamp and the friction head.
3) Place the specimen clamp head onto the friction platform, with the core shaft inserted through the bearing onto the specimen clamp head; then add a 395 g weight (the load generated by the 395 g weight plus the weight of the 200 g metal clamp head is 583.1 CN).
4) Set the rotation speed of the instrument to 20 rpm with a total of 15 rotations. After completing the setting, click the “Start” button to initiate the operation of the instrument, and once the set number of tests for the instrument is completed, the instrument will stop. Inspect the fuzzing condition of the lower-layer abrasive material and determine its abrasion resistance level as L (good abrasion resistance), M (moderate abrasion resistance), or H (poor abrasion resistance) based on the fuzzing condition.

Using the aforementioned test items and methods, the non-woven fabric wipes produced in Embodiments 1 and 2 and a conventional non-woven fabric wipe are subjected to testing and evaluation respectively, where the conventional non-woven fabric wipe is a wood pulp melt-blown non-woven fabric with two side surface layers being polypropylene melt-blown fiber webs and the middle layer being wood pulp.

| | Gram | Powder drop | Abrasion |
|---|---|---|---|
| | weight | rate | resistance |
| | Unit | | |
| Test item | g/m² | % | / |
| Wood pulp melt-blown non- | 65 | 0.25 | M |
| woven fabric | | | |
| Sample of Embodiment 1 | 65 | 0.13 | L |
| Sample of Embodiment 2 | 65 | 0.14 | L |

If the aforementioned technical solutions are adopted, since the non-woven fabric wipes 1, 2 of the present invention is composed of five layers of fiber webs, where the third layer 13, 23 is wood pulp fibers, the first layer 11, 21, the second layer 12, 22, the fourth layer 14, 24, and the fifth layer 15, 25 are all composed of melt-blown fibers, and the melt-blown fibers of the second layer 12, 22, the third layer 13, 23 and the fourth layer 14, 24 positioned at the middle position are partially interwoven with the wood pulp fibers; when the melt-blown fiber webs of the second layer 12, 22 and the fourth layer 14, 24 are consolidated, the portions interwoven with the wood pulp fibers will be consolidated together with the interwoven wood pulp fibers, and the consolidated wood pulp fibers are positioned at the upper and lower surfaces of the third layer 13, 23, such that a protective web that prevents the wood pulp fibers from moving is formed, and the melt-blown fiber webs of the first layer 11, 21, the second layer 12, 22, the fourth layer 14, 24 and the fifth layer 15, 25 positioned outside the third layer 13, 23 protect the wood pulp fibers layer by layer, thereby preventing the phenomenon of “linting”. In addition, the melt-blown fiber webs of the first layer 11, 21 and the fifth layer 15, 25 are compounded with the multilayer structured fiber web with compounding included angles θ12, θ13, θ22, and θ23 all being 90°, such that the melt-blown fiber webs of the first layer 11, 21 and the fifth layer 15, 25, before formation, are perpendicular to the multilayer structured fiber web, and the melt-blown fiber webs of the first layer 11, 21 and the fifth layer 15, 25 are only perpendicularly laid on the surface of the multilayer structured fiber web. The melt-blown fibers are not in contact with the non-melt-blown fibers in the multilayer structured fiber web, and therefore the formed melt-blown fiber webs only contain melt-blown fibers, which makes the fiber webs more compact and uniform during consolidation, and is more favorable for the protection of the wood pulp short fibers in the third layer 13, 23. If the above solutions of the present invention are adopted, Embodiments 1 and 2 have powder drop rates of 0.13% and 0.14% respectively and an “L” abrasion resistance grade (good abrasion resistance), while the conventional non-woven fabric wipe has a powder drop rate of 0.25% and a “M” abrasion resistance grade (moderate abrasion resistance), such that the powder drop rate of the non-woven fabric wipe is effectively reduced, and the abrasion resistance is improved. Meanwhile, the fiber deniers of the first layer 11, 21 and the fifth layer 15, 25 are 0.2-0.4 denier, and the fibers are finer. A more compact and uniform fiber structure can be formed to prevent flake dropping and linting, and the overall soft touch of the non-woven fabric wipe is also improved. The melt-blown fiber deniers of the second layer 12, 22 and the fourth layer 14, 24 are 0.5-2.0 denier, and the fibers are thicker and are easier to interweave with the wood pulp.

In Embodiment 2, the melt-blown fiber surface of the first layer 21 positioned at the surface layer of the non-woven fabric wipe 2 is further subjected to a hydrophilic post-treatment, such that the first layer 21 of the non-woven fabric wipe 2 has hydrophilicity, and the overall water absorption of the non-woven fabric wipe 2 is enhanced. It is more conducive to removing water stains and dirt when the non-woven fabric wipe 2 is used, rendering a stronger cleaning function. In a post-treatment procedure, a functional coating, such as weak lipophilic agent, jojoba oil, chamomile, aloe, shea butter and other surfactants, can be applied by spray coating or roll coating to improve the skin-friendly performance of the non-woven fabric wipe 2. Meanwhile, the fiber thicknesses and densities of the first layer and the fifth layer can be adjusted to meet different requirements of clients and to be applied in different fields.

1. A non-woven fabric wipe having a layered fiber web structure, comprising a first layer, a second layer, a third layer, a fourth layer and a fifth layer which are sequentially arranged; the first layer, the second layer, the fourth layer and the fifth layer of the non-woven fabric wipe are all melt-blown fiber webs mainly-composed of melt-blown fibers, and the third layer of the non-woven fabric wipe is a wood pulp fiber web mainly composed of wood pulp fibers, wherein the first layer and the fifth layer each has a melt-blown fiber denier of 0.2-0.4 denier, the second layer and the fourth layer each has a melt-blown fiber denier of 0.5-2.0 denier, and a weight percentage of the third layer to the non-woven fabric wipe is ≥65%; the melt-blown fibers in the melt-blown fiber webs of the second layer and the fourth layer are partially interwoven into the wood pulp fiber web of the third layer adjacent thereto.

2. The non-woven fabric wipe of claim 1, wherein the melt-blown fibers are polyolefin fibers, polyester fibers, polyamide fibers, polyurethane fibers, polylactic acid fibers, or a mixture thereof.

3. The non-woven fabric wipe of claim 1, wherein the melt-blown fibers are single-component melt-blown fibers, bi-component melt-blown fibers, or melt-blown fibers with a blend of the single-component melt-blown fibers and the bi-component melt-blown fibers.

4. non-woven fabric wipe of claim 3, wherein the bi-component melt-blown fibers are composed of fibers having a melting point difference of 20° C. or more, and each of the fibers having low-melting point resin at its surface.

5. The non-woven fabric wipe of claim 1, wherein the wood pulp fiber web of the third layer is composed of a blend of wood pulp fibers, viscose fibers, single-component or bi-component chemical fibers, or mixed fibers thereof.

6. The non-woven fabric wipe of claim 1, wherein the wood pulp fiber web of the third layer is composed of a blend of wood pulp fibers, hot-melt adhesive substances, super absorbent resins, or a blend thereof.

7. The non-woven fabric wipe of claim 1, wherein an embossed area is formed on a surface of the non-woven fabric wipe.

8. The non-woven fabric wipe of claim 1, wherein a hydrophilic coating is provided on an upper surface of the melt-blown fiber web of the first layer.

9. (canceled)

10. (canceled)

11. (canceled)

12. (canceled)

13. (canceled)
